(19) United States
(12) Patent Application Publication
(10) Pub. No.: US 2009/0103048 A1
(43) Pub. Date: Apr. 23, 2009

(54) METHOD AND SYSTEM FOR PUPIL DETECTION

(75) Inventor: Shuichiro Tsukiji, Santa Clara, CA (US)

Correspondence Address: OSHA LIANG L.L.P., TWO HOUSTON CENTER, 909 FANNIN, SUITE 3500, HOUSTON, TX, 77010, US

(73) Assignee: OMRON SILICON VALLEY, Santa Clara, CA (US)

(21) Appl. No.: 11/874,040

(22) Filed: Oct. 17, 2007

(51) Int. Cl. A61B 3/14

(52) U.S. Cl. 351/206; 351/246

(57) ABSTRACT

A method for detecting a location of a pupil. The method involves projecting a modulated light at a first phase towards a face from a lighting source located near an optical axis of a modulated light camera, concurrently projecting a modulated light at a second phase towards the face from a lighting source located off the optical axis of the modulated light camera, where the first phase and the second phase are different; receiving a light reflected from the face; and generating an image from the light reflected from the face, where the image indicates the location of the pupil.

### BACKGROUND

Pupil detection is a process of measuring the point of gaze or the location of the pupil relative to the face. Pupil detection can be used for many different applications. For example, pupil detection can be used for cognitive studies, medical research, computer applications, translation process research, vehicle simulators in-vehicle research, training simulators, virtual reality, primate research, sports training, web usability, advertising, marketing, communication for the disabled, and automotive engineering.

Over the years, various methods for pupil detection have been developed. FIGS. 1A-1C shows one conventional method for pupil detection. This conventional method leverages the use of light entering an eyeball (126) through a pupil (125) and capturing light reflected, off the retina, out of the eyeball (126) through the pupil (125). As shown in FIG. 1, this method includes two light sources, i.e., light source A (110) off the optical axis (106) of camera (105) and light source B (115) near the optical axis (106) of the camera (105).

The two light sources emit light in alternating frames of the camera (105). As shown in FIG. 1A, in the first frame of the camera (105), light source A (110), which is off the optical axis (106), is turned off. Further, light source B (115), which is near the optical axis (106) of the camera (105), is turned on to emit light B (116) onto the face (120), including the pupil (125). Additionally, a portion of light B (116) enters the eyeball (126) through the pupil (125) and results in a lighted retinal area B (128). Because light source B (115) is close to the optical axis (106) of the camera (105), the lighted retinal area B (128) overlaps with the viewable portion of retina (127), i.e., the portion of the retina that creates a retinal reflection toward the camera (105) when lighted. Accordingly, reflected light B (117) captured by the camera (105) includes a retinal reflection and results in a light pupil image (130) generated by the camera (105). The light pupils in the light pupil image (130) may also be referred to as a “red-eye” effect.

FIG. 1B shows a second frame of the camera (105), which alternates with the first frame of the camera (105) shown in FIG. 1A. As shown in FIG. 1B, in the second frame of the camera (105), light source B (115), which is near the optical axis (106), is turned off. However, light source A (110), which is off the optical axis (106) of the camera (105), is turned on to emit light A (111) onto the face (120) including the pupil (125). Additionally, a portion of light A (111) enters the eyeball (126) through the pupil (125) and results in a lighted retinal area A (129). Because light source A (110) is off the optical axis (106) of the camera (105), the lighted retinal area A (129) does not overlap with the viewable portion of retina (127) described above. Accordingly, reflected light A (112) captured by the camera (105) does not include a retinal reflection and results in a dark pupil image (135) generated by the camera (105).

By using alternating frames in correspondence with alternating lights, the method of pupil detection is able to generate a dark pupil image (135) and a light pupil image (130) in exactly two time frames of the camera. Thereafter, as shown in FIG. 1C, the light pupil image (130) and the dark pupil image (135) are analyzed in combination to find a differential image (140) which is used to detect the pupil (125).

However, the use of multiple frames may result in distortion between the light pupil image (130) and the dark pupil image (135) when the face or the camera moves between the first frame and the second frame of the camera (105).

### SUMMARY

In general, in one aspect, one or more embodiments of the invention relate to a method for detecting a location of a pupil, comprising: projecting a first modulated light at a first phase towards a face from a first lighting source located near an optical axis of a modulated light camera; concurrently projecting a second modulated light at a second phase towards the face from a second lighting source located off the optical axis of the modulated light camera, wherein the first phase and the second phase are different; receiving a light reflected from the face; and generating an image from the light reflected from the face, wherein the image C indicates the location of the pupil.

In general, in one aspect, one or more embodiments of the invention relate to a method for detecting a location of an pupil, comprising: projecting a first modulated light towards a face from a first lighting source located close to an optical axis of a modulated light camera at a first wavelength resulting in a first light reflection from the face; concurrently projecting a second modulated light towards the face from a second lighting source located off the optical axis of the modulated light camera at a second wavelength resulting in a second light reflection from the face, wherein the first wavelength and the second wavelength are different; receiving the first light reflection by a first modulated light camera, wherein the second light reflection is filtered out; receiving the second light reflection by a second modulated light camera, wherein the first light reflection is filtered out; generating an image using a first output from the first modulated light camera and a second output from the second modulated light camera, wherein the image indicates a location of the pupil.

In general in one aspect, one or more embodiments of the invention relate to a system for detecting a location of a pupil, comprising: a first lighting source disposed near an optical axis of a modulated light camera that projects a first modulated light at a first phase towards a face; a second lighting source disposed off the optical axis of the modulated light camera that projects a second modulated light at a second phase towards the face concurrently with the first lighting source projecting the first modulated light towards the face, wherein the first phase and the second phase are different; wherein the modulated light camera receives the light reflected from the face and generates an image from the light reflected from the face; and wherein the image indicates a location of the pupil.

In general, in one aspect, one or more embodiments of the invention relate to a program stored on computer readable medium comprising instructions for causing a system to detect a location of a pupil, the instructions for: projecting a first modulated light at a first phase towards a face from a first lighting source located near an optical axis of a modulated light camera; concurrently projecting a second modulated light at a second phase towards the face from a second lighting source located off the optical axis of the modulated light camera, wherein the first phase and the second phase are different; receiving a light reflected from the face; and generating an image from the light reflected from the face, wherein the image indicates the location of the pupil.

Other aspects and advantages of the invention will be apparent from the following description and the appended claims.

### BRIEF DESCRIPTION OF DRAWINGS

FIGS. 1A-1C show a prior method of pupil detection.

FIGS. 2A and 2B show a system for pupil detection in accordance with one or more embodiments of the invention.

FIG. 3 shows a diagram in accordance with one or more embodiments of the invention.

FIGS. 4 and 5 show a flow chart in accordance with one or more embodiments of the invention.

FIG. 6 shows a system in accordance with one or more embodiments of the invention.

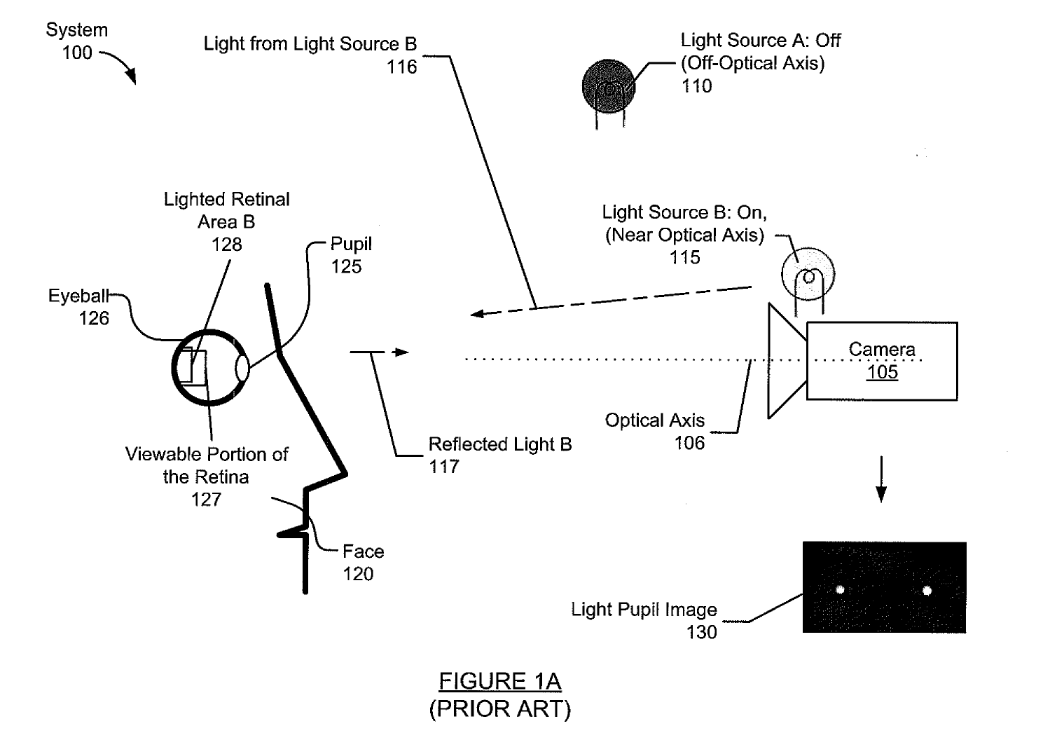

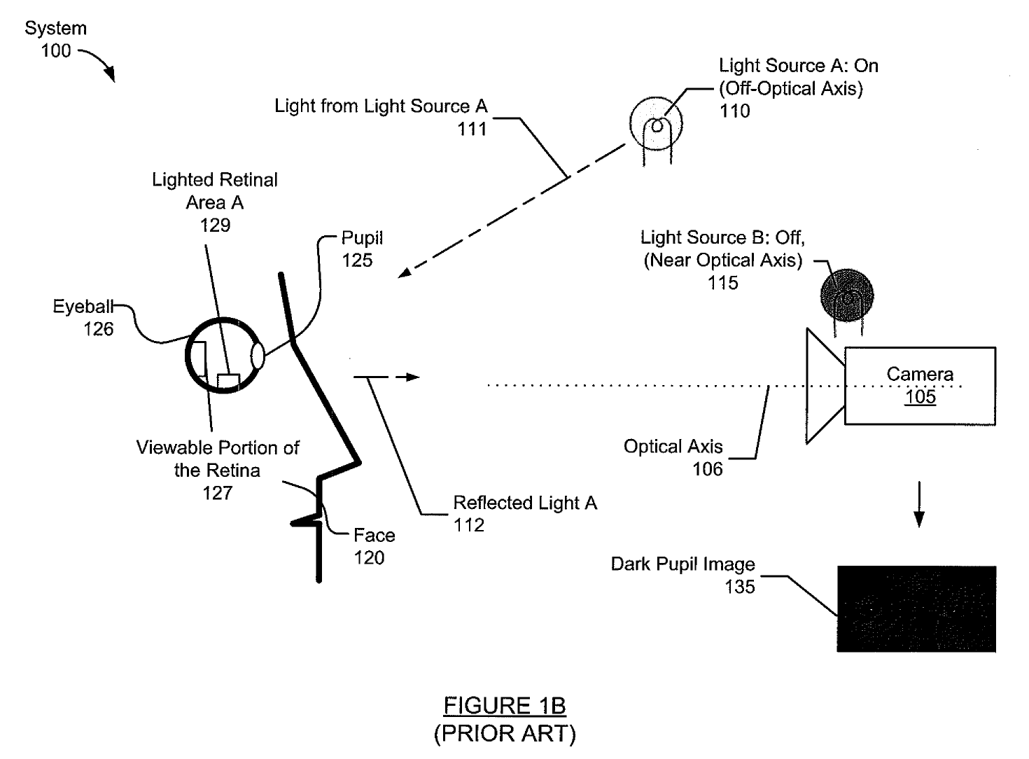

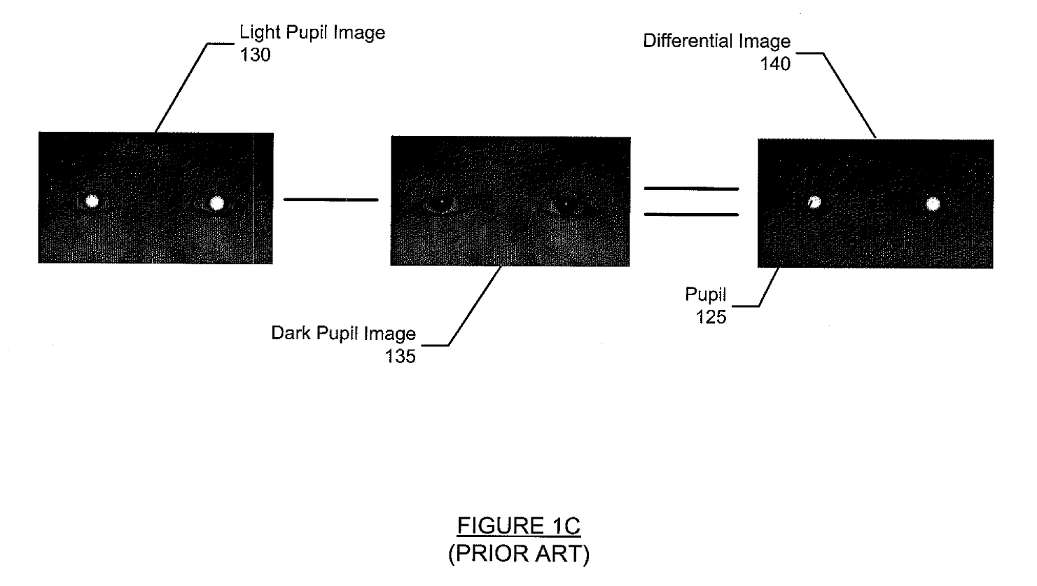

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, one or more embodiments of the invention are directed toward a method for detecting a location of a pupil. Specifically, one or more embodiments of the invention allow for detecting a location of a pupil in a single time frame of a modulated light camera.

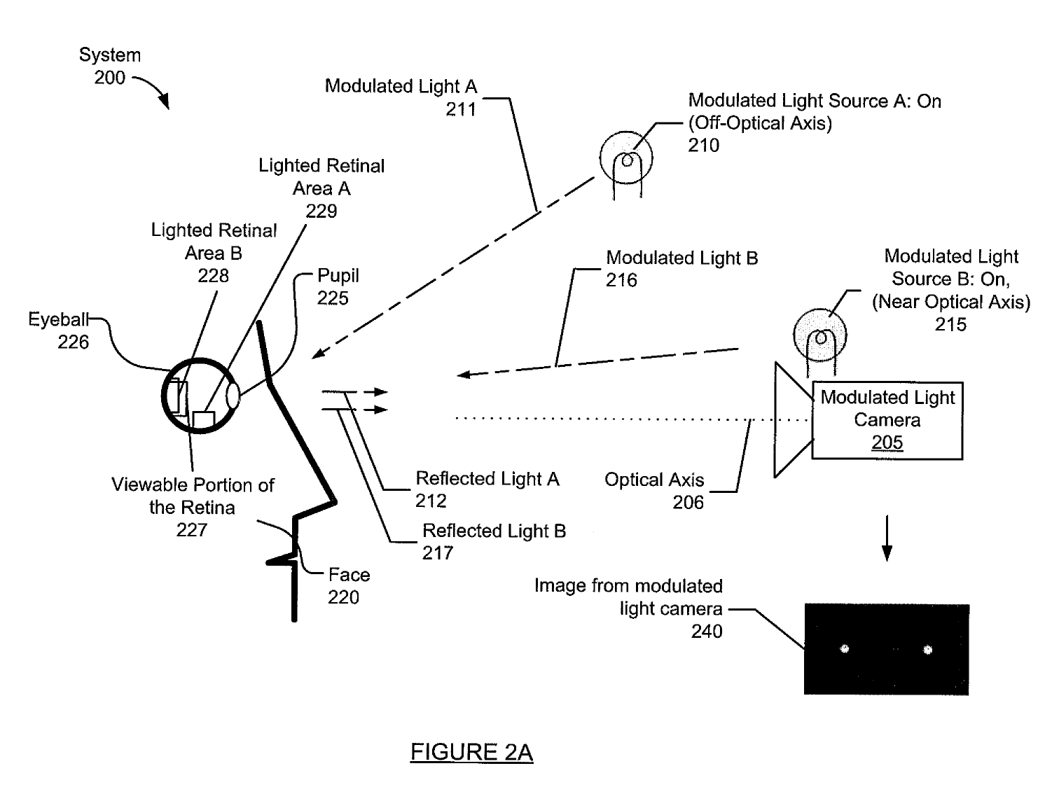

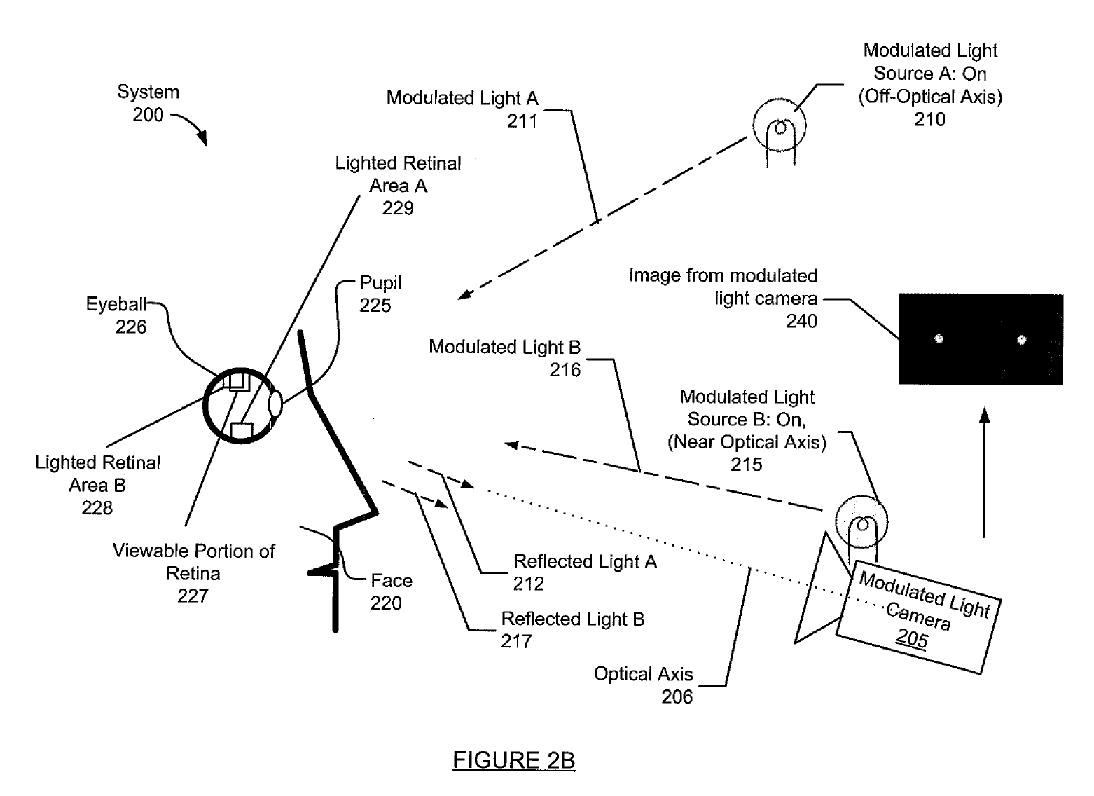

As shown in FIG. 2A, the system (200) includes multiple light sources (e.g., modulated light source A (210) and modulated light source B (215)) and at least one modulated light camera (205). In one or more embodiments of the invention, natural or environmental light sources may also be included (not shown).

In one or more embodiments of the invention, modulated light source A (210) and modulated light source B (215) each correspond to at least one temporal light modulator (TLM). A TLM is a device that includes functionality to vary a property of light as a function of time in accordance with a varying signal which can be of any energy form, to generate a modulated light. Such properties may include, but are not limited to, intensity (i.e., amplitude), frequency, phase, and/or polarization. The variation may be imparted using elasto-optic, magneto-optic, acousto-optic, and/or other suitable modulation mechanisms. An elasto-optic modulation may include varying a property of a light wave by applying mechanical stress. A magneto-optic modulation may include varying a property of a light wave by applying a magnetic field. Lastly, an acousto-optic modulation may include varying a property of a light wave by applying modulated sonic (acoustic) waves. The various modulation mechanisms may also be used in combination to project modulated light. Furthermore, a TLM may use liquid crystal (e.g., liquid crystal modulators switched by either thin-film transistors, or silicon backplanes), pixilated crystal (e.g., pixilated crystal of aluminum garnet switched by an array of magnetic coils using the magneto-optic effect), deformable mirrors (e.g., a two-dimensional array of movable mirrors, where each mirror is controlled by electrostatic force, deflecting light out depending on the tilt of the mirror), and/or other suitable devices to project modulated light. In one or more embodiments of the invention, the modulated light corresponds to infrared light, which is non-invasive and invisible to the human eye due to the wavelength of the light. One skilled in the art will appreciate that other lights (e.g., of different wavelengths) may also be used in accordance with one or more embodiments of the invention.

In one or more embodiments of the invention, modulated light source A (210) is located off the optical axis (206) of the modulated light camera (205) and includes functionality to project modulated light A (211) towards a face (220) that results in a reflection of modulated light, i.e., reflected light A (212) from the face (220). Further, the modulated light source A (210) includes functionality to project modulated light A (211) directly towards (and through) the pupil (225) that results in a lighted retinal area A (229) inside the eyeball (226). A lighted retinal area corresponds to a lighted portion of the retina within the eyeball (226) that receives external light through the pupil. Because modulated light source A (210) is off the optical axis (206) of the modulated light camera (205), the lighted retinal area A (229) does not overlap with the viewable portion of retina (227), described above, that corresponds to the modulated light camera (205). Accordingly, the reflected light A (212), projecting towards the modulated light camera (205), is a light reflection of the face (220) without a substantial retinal reflection. In one or more embodiments of the invention, the modulated light source A (210) may be adjusted to change the angle between modulated light A (211) and the optical axis (206) with respect to the pupil (225) such that the retinal reflection is reduced or eliminated.

In one or more embodiments of the invention, modulated light source B (215) is located near the optical axis (206) of the modulated light camera (205) and includes functionality to project modulated light B (216) towards the face (220) that results in a reflection of modulated light, i.e., reflected light B (217) from the face (220). The modulated light source B (215) includes functionality to project modulated light B (216) directly towards (and through) the pupil (225) that results in a lighted retinal area B (228) inside the eyeball (226). Because modulated light source B (215) is near the optical axis (206) of the camera (205), the lighted retinal area B (228) overlaps with the viewable portion of retina (227), described above, that corresponds to the modulated light camera (205). Accordingly, the reflected light B (217), projecting towards the modulated light camera (205), is a light reflection of the face (220) including a retinal reflection from lighted retinal area B (228). In one or more embodiments of the invention, the modulated light source B (215) may be adjusted to change the angle between modulated light B (216) and the optical axis (206) with respect to the pupil (225) such that the retinal reflection in reflected light B (217) is increased. As shown in FIG. 2B, in one or more embodiments of the invention, the modulated light camera (205) may be located anywhere with respect to the pupil (225), such that the modulated light source B (216) is near the optical axis (206) of the modulated light camera (205) resulting in capture of reflected light B (217) including a retinal reflection from lighted retinal area B (228).

Continuing with reference to FIG. 2A, in one or more embodiments of the invention, modulated light source A (210) and modulated light source B (215) are controlled, directly or indirectly, by a host computer (not shown). For example, a TLM driver (not shown) may be used to interface with the host computer and the TLM. The TLM driver may accept instructions and patterns from the host computer, and write the pattern onto the TLM to modulate the incoming light.

In one or more embodiments of the invention, the modulated light camera (205) corresponds to an imaging device configured to receive the reflected light (i.e., reflected light A (212) and reflected light B (217)) from the face (220) as a result of the projection of modulated light A (211) and modulated light B (216), respectively, onto the face (220). The modulated light camera (205) may include functionality to filter out any non-modulated light, e.g., light from environmental sources (not shown), non-retinal portions of reflected light A (212) and non-retinal portions of reflected light B (217) that become non-modulated light due to the phase difference between reflected light A (212) and reflected light B (217). Furthermore, in one or more embodiments of the invention, the modulated light camera (205) may use optical filters to limit incoming light to a predetermined wavelength range. The modulated light camera (205) may be configured to filter out any wavelengths not coinciding with the approximate wavelengths of modulated light A (211) and modulated light B (216). For example, if modulated light A (211) and modulated light B (216) correspond to infrared light, the modulated light camera (205) may exclude any light out of a range coinciding with infrared light. In accordance with one or more embodiments of the invention, the range of the wavelengths allowed by adjusted continuously or discretely for improved pupil detection. Furthermore, in one or more embodiments of the invention, the modulated light camera (205) includes functionality to generate an image (240) using non-filtered out portions of reflected light A (212) and reflected light B (217). The image (240) generated by the modulated light camera (205) may be used to determine the location of the pupil (225) with respect to the face (220) and/or the point of gaze.

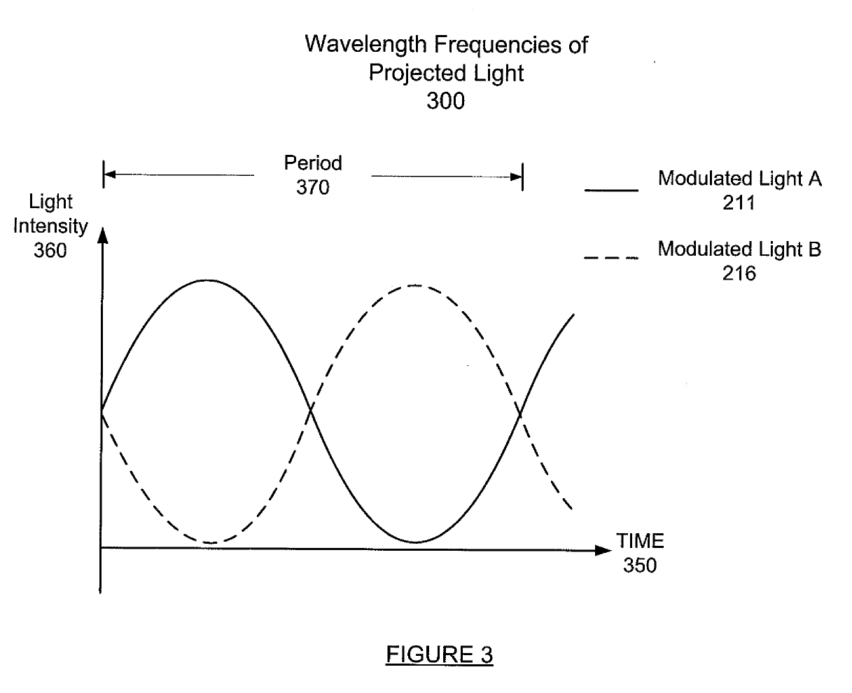

In one or more embodiments of the invention, the phase difference between modulated light A (211) and modulated light B (216) is approximately 180°, as shown in FIG. 3, which results in a similar phase difference of approximately 180° between corresponding reflected light A (212) and reflected light B (217). A non-pupil portion of reflected light A (212) and a similar non-pupil portion of reflected light B (217) reduce or eliminate the modulation of each other due to the phase difference of approximately 180°. One skilled in the art will appreciate, that the phase difference of modulated light A (211) and modulated light B (216) may be adjusted (e.g., to any range between 0° and 180°), based on the varying distance of each light source from the face, to ensure that reflected light A (212) and reflected light B (217) are at approximately a 180° phase difference. The phase difference between reflected light A (212) and reflected light B (217) does not result in a reduction or elimination in the modulation of non-similar portions (e.g., the retinal reflection of reflected light B (217)).

Although light intensity as a function of time is shown as sinusoidally modulated for modulated light A (211) and for modulated light B (216), one skilled in the art will appreciate that alternate modulation schemes may be implemented that result in approximately a 180° phase difference between reflected light A (212) and reflected light B (217). For example, modulated light A (211) and modulated light B (216) may be modulated alternatively at a constant high intensity and a constant low intensity. In another embodiment of the invention, multiple sets of modulated lights may be used where each set results in a 180° phase difference in the corresponding reflected light and each set includes one light source near the optical axis (206) of the modulated light camera (205) and one light source off the optical axis (206) of the modulated light camera (205). Furthermore, although reflected light A (212) and reflected light B (217) are shown as having the same amplitude (i.e., light intensity), one skilled in the art will appreciate that the amplitude of reflected light A (212) may be different than the amplitude of reflected light B (217). In addition, the amplitude of each reflected light may vary over time.

Furthermore, in one or more embodiments of the invention, multiple modulated light cameras may be used where each camera corresponds to a particular light source and filters in light based on the frequency of the corresponding light source. Accordingly, each camera may generate an image based on the reflected light from the corresponding light source. Thereafter, the images from the multiple modulated light cameras may be analyzed in combination to determine the point of gaze or the location of the pupils with respect to the face.

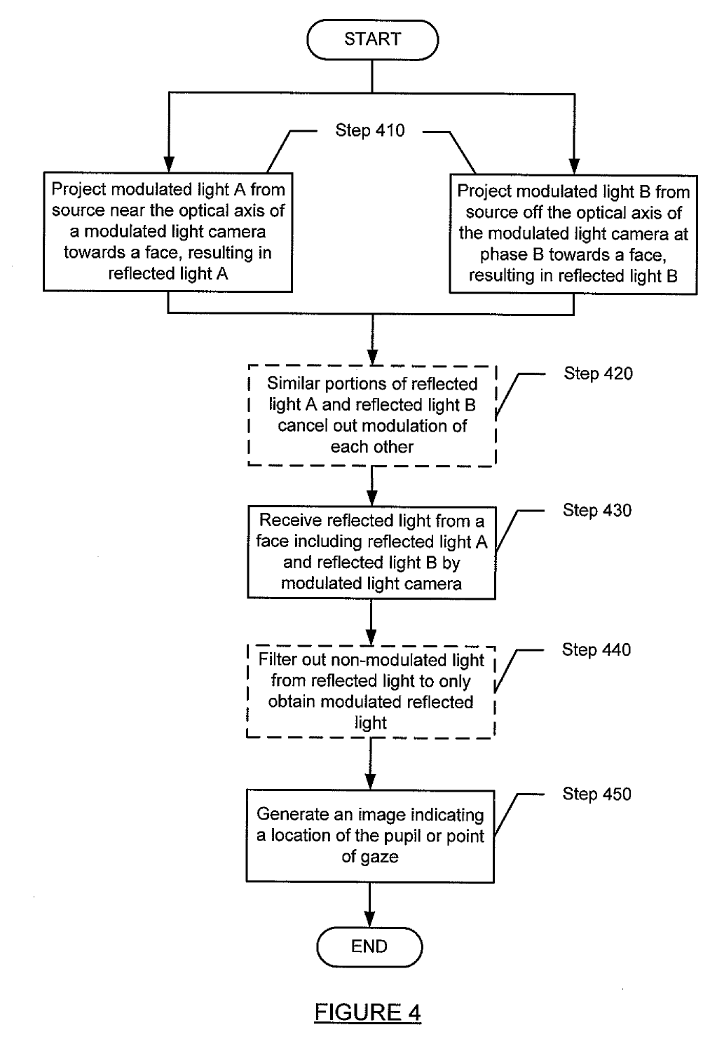

FIG. 4 shows a flow chart in accordance with one or more embodiments of the invention. In one or more embodiments of the invention, one or more of the steps described below may be omitted, repeated, and/or performed in a different order.

Initially, modulated light A, from a source near the optical axis of a modulated light camera, is projected towards a face resulting in reflected light A reflected from the face towards the modulated light camera. Concurrently, modulated light B from a source off the optical axis of the modulated light camera is projected towards the face resulting in reflected light B reflected from the face towards the modulated light camera (Step 410). Modulated light A and modulated light B may be projected using different or similar light modulation technologies, described above. In addition, non-modulated environmental light may also be concurrently projected towards the face. Further, modulated light A and modulated light B may be projected continuously or in discrete segments of time.

In one or more embodiments of the invention, the phase, amplitude, frequency, and/or polarization of modulated light A and modulated light B may be controlled by one or more computers, such that the phase difference between reflected light A (i.e., light reflection as a result of projected modulated light A) and reflected light B (i.e., light reflection as a result of projected modulated light B) is approximately a 180°. As a result of the phase difference between reflected light A and reflected light B, similar portions of reflected light A and reflected light B may reduce or cancel out the modulation of one another (Step 420). Non-similar portions of reflected light A and reflected light B maintain modulation because the non-similarity prevents the phase difference from having an effect on the modulation of each. The projection of modulated light A from a source near the optical axis of the modulated light camera towards the face results in a retinal reflection through the pupil in reflected light A. The projection of modulated light B from a source off the optical axis of the modulated light camera towards the face does not result in a substantive retinal reflection in reflected light B. Accordingly, because reflected light A includes a retinal reflection and reflected light B does not include a retinal reflection, reflected light A and reflected light B may be non-similar and furthermore, a portion of reflected light B corresponding to the retinal reflection maintains modulation.

In one or more embodiments of the invention, at least a portion of light is received by the modulated light camera (Step 440). Any non-modulated light (e.g., environmental light and similar portions of reflected light A and reflected light B that have become non-modulated) are filtered out by the modulated light camera before processing (Step 440). Thereafter, the remaining modulated light, that is not filtered out, is analyzed to generate an image which may be used to determine a location of the pupil or a point of gaze (Step 450). The image may also be modified to better determine the location of the pupil or the point of gaze. For example, bright points that cannot correspond to a pupil due to size or shape may be rejected as noise. The images may also be modified based on variations and differences in the amplitude of the modulated lights. In another example, temporal smoothing may be applied to improve determination of pupil locations or gaze points.

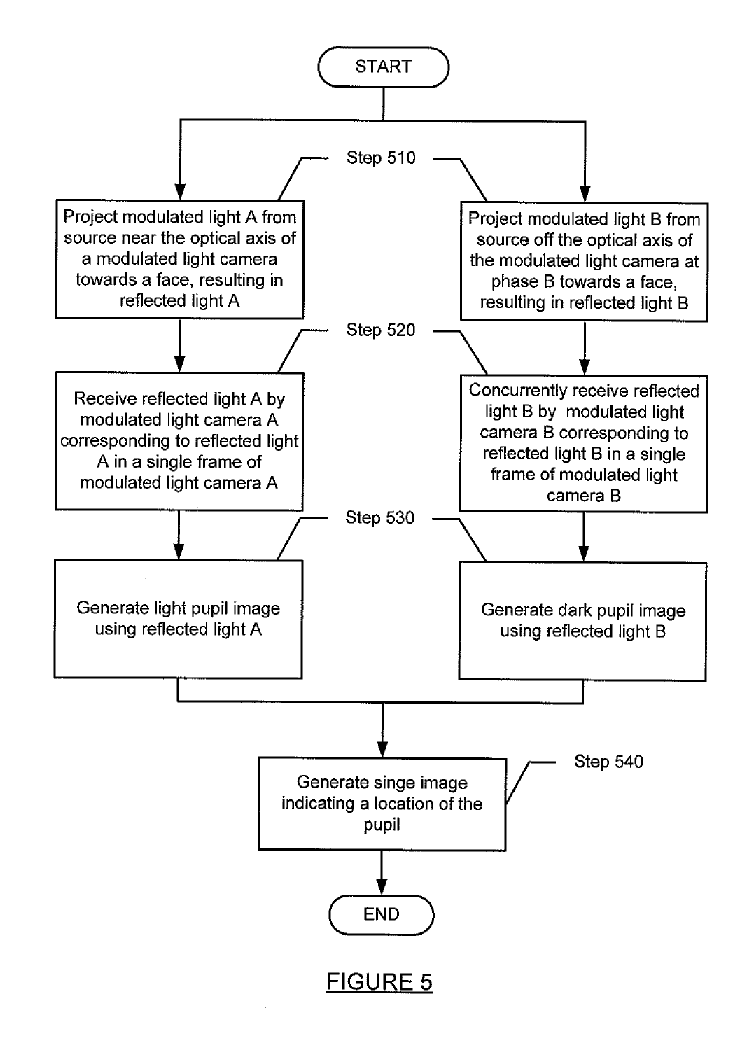

FIG. 5 shows a flow chart in accordance with one or more embodiments of the invention. In one or more embodiments of the invention, one or more of the steps described below may be omitted, repeated, and/or performed in a different order.

Initially, modulated light A and modulated light B are projected toward a face (Step 510). Step 510 is essentially the same as Step 410 described above. Next, reflected light A is received by modulated light camera A in a single frame of modulated light camera A and other light (e.g., reflected light B and environmental light) is filtered out by modulated light camera A. Concurrently, reflected light B is received by modulated light camera B in a single frame of modulated light camera B and other light (e.g., reflected light A and environmental light) is filtered out by modulated light camera B (Step 520). For example, modulated light A and modulated light B may be projected at different wavelengths and each modulated light camera may use filters for limiting the light received to wavelengths for corresponding modulated light, respectively.

Thereafter, each modulated light camera generates an image using the respective reflected light received from the face, in accordance with one or more embodiments of the invention (Step 530). Specifically, in this example, modulated light camera A, which received reflected light A, generates a light pupil image since reflected light A includes a retinal reflection due to the location of the modulated light source A. Modulated light camera B, which received reflected light B, generates a dark pupil image since reflected light B did not include a retinal reflection due to the location of modulated light source B.

Next, the images generated in Step 530 are combined to generate a single image that indicates the point of gaze or the location of the pupils relative to the face (Step 540). In one or more embodiments of the invention, the single image is generated using a differential between each of the prior images generated by the modulated light cameras. In one or more embodiments of the invention, the single image is analyzed using image analysis techniques to estimate pupil center, radius, and contours. Furthermore, the image may also be used to estimate the iris radius and contours.

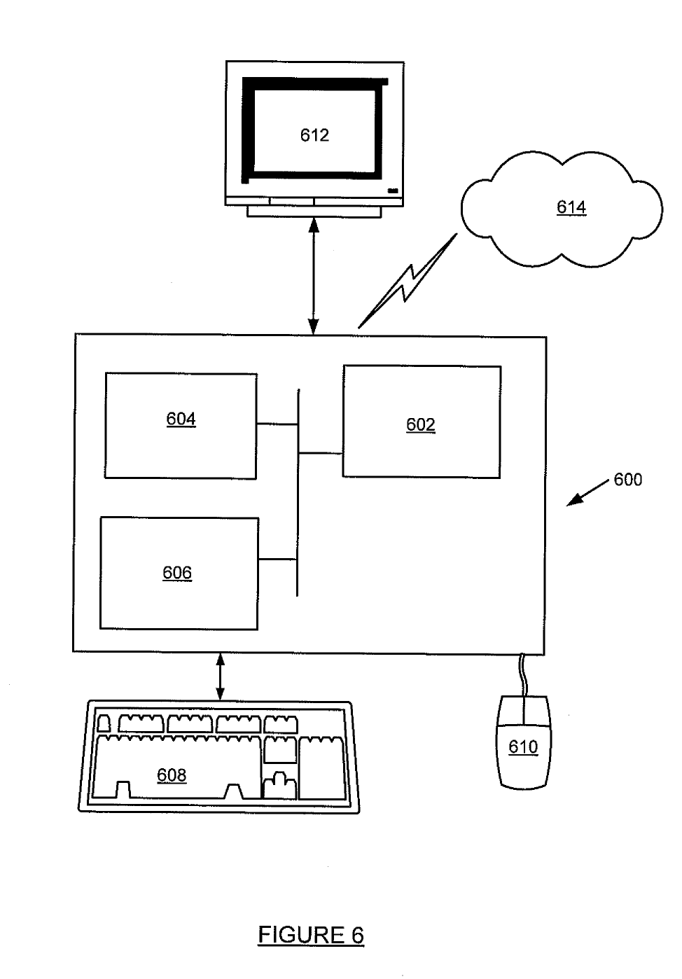

The invention may be implemented on virtually any type of computer regardless of the platform being used. For example, as shown in FIG. 6, a computer system (600) includes a processor (602), associated memory (604), a storage device (606), and numerous other elements and functionalities typical of today's computers (not shown). The computer (600) may also include input means, such as a keyboard (608) and a mouse (610), and output means, such as a monitor (612). The computer system (600) is connected to a LAN or a WAN (e.g., the Internet) (614) via a network interface connection. Those skilled in the art will appreciate that these input and output means may take other forms.

Further, those skilled in the art will appreciate that one or more elements of the aforementioned computer system (600) may be located at a remote location and connected to the other elements over a network. Further, the invention may be implemented on a distributed system having a plurality of nodes, where each portion of the invention (e.g., object store layer, communication layer, simulation logic layer, etc.) may be located on a different node within the distributed system. In one embodiment of the invention, the node corresponds to a computer system. Alternatively, the node may correspond to a processor with associated physical memory. The node may alternatively correspond to a processor with shared memory and/or resources. Further, software instructions to perform embodiments of the invention may be stored on a computer readable medium such as a compact disc (CD), a diskette, a tape, a file, or any other computer readable storage device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for detecting a location of a pupil, comprising:
projecting a first modulated light at a first phase towards a face from a first lighting source located near an optical axis of a modulated light camera;
concurrently projecting a second modulated light at a second phase towards the face from a second lighting source located off the optical axis of the modulated light camera, wherein the first phase and the second phase are different;
receiving a light reflected from the face; and
generating an image from the light reflected from the face,
wherein the image indicates the location of the pupil.

2. The method of claim 1, wherein the image indicates a location of a pupil with respect to the face.

3. The method of claim 1, wherein the light reflected from the face comprises a first reflected light corresponding to the first modulated light and a second reflected light corresponding to the second modulated light, and wherein the first reflected light and the second reflected light have approximately a 180° phase difference.

4. The method of claim 1, wherein the light reflected from the face is received in a single frame of the modulated light camera.

5. The method of claim 1, further comprising filtering out non-modulated light reflecting from the face prior to generating the image.

6. The method of claim 1, wherein projection of the first modulated light from the first light source near the optical axis of the modulated light camera results in a retinal reflection, and wherein the retinal reflection is comprised in the light reflected from the face.

7. The method of claim 6, wherein the projection of the second modulated light from the second light source off the optical axis of the modulated light camera does not result in a retinal reflection.

8. The method of claim 7, wherein the light reflected from the face further comprises a non-retinal reflection resulting from the projection of the first modulated light and a non-retinal reflection resulting from the projection of the second modulated light, and wherein a portion of the non-retinal reflection resulting from the projection of the first modulated light and a portion of the non-retinal reflection resulting from the projection of the second modulated light become non-modulated due to the phase difference between the first phase and the second phase.

9. A method for detecting a location of an pupil, comprising:
projecting a first modulated light towards a face from a first lighting source located close to an optical axis of a modulated light camera at a first wavelength resulting in a first light reflection from the face;
concurrently projecting a second modulated light towards the face from a second lighting source located off the optical axis of the modulated light camera at a second wavelength resulting in a second light reflection from the face, wherein the first wavelength and the second wavelength are different;
receiving the first light reflection by a first modulated light camera, wherein the second light reflection is filtered out;
receiving the second light reflection by a second modulated light camera, wherein the first light reflection is filtered out;
generating an image using a first output from the first modulated light camera and a second output from the second modulated light camera, wherein the image indicates a location of the pupil.

10. A system for detecting a location of a pupil, comprising:
a first lighting source disposed near an optical axis of a modulated light camera that projects a first modulated light at a first phase towards a face;
a second lighting source disposed off the optical axis of the modulated light camera that projects a second modulated light at a second phase towards the face concurrently with the first lighting source projecting the first modulated light towards the face,
wherein the first phase and the second phase are different;
wherein the modulated light camera receives the light reflected from the face and generates an image from the light reflected from the face; and
wherein the image indicates a location of the pupil.

11. The system of claim 10, wherein the image indicates a location of a pupil with respect to the face.

12. The system of claim 10, wherein the light reflected from the face comprises a first reflected light at the first phase corresponding to the first modulated light and a second reflected light at the second phase corresponding to the second modulated light, and wherein the first reflected light and the second reflected light have approximately a 180° phase difference.

13. The system of claim 10, wherein the image is generated from the light reflected in a single frame of the modulated light camera.

14. The system of claim 10, wherein the modulated light camera filters out non-modulated light reflected from the face prior to generating the image.

15. The system of claim 10, wherein projection of the first modulated light from the first light source near the optical axis of the modulated light camera results in a retinal reflection, and wherein the retinal reflection is comprised in the light returned from the face.

16. The system of claim 10, wherein projection of the second modulated light from the second light source does not result in a retinal reflection.

17. The system of claim 16, wherein a portion of the light returned from the face becomes non-modulated due to the phase difference between the first modulated light and the second modulated light.

18. A program stored on computer readable medium comprising instructions for causing a system to detect a location of a pupil, the instructions for:
projecting a first modulated light at a first phase towards a face from a first lighting source located near an optical axis of a modulated light camera;
concurrently projecting a second modulated light at a second phase towards the face from a second lighting source located off the optical axis of the modulated light camera, wherein the first phase and the second phase are different;
receiving a light reflected from the face; and
generating an image from the light reflected from the face,
wherein the image indicates the location of the pupil.

19. The computer readable medium of claim 18, wherein the image indicates a location of a pupil with respect to the face.

20. The program stored on computer readable medium of claim 22, wherein the light reflected from the face comprises a first reflected light corresponding to the first modulated light and a second reflected light corresponding to the second modulated light, and wherein the first reflected light and the second reflected light have approximately a 180° phase difference.

21. The computer readable medium of claim 18, wherein the light reflected from the face used for generating the image is received in a single frame of the modulated light camera.

22. The computer readable medium of claim 18, further comprising instructions for filtering out non-modulated light reflecting from the face prior to generating the image.

23. The computer readable medium of claim 18, wherein projection of the first modulated light from the first light source near the optical axis of the modulated light camera results in a retinal reflection, and wherein the retinal reflection is comprised in the light reflected from the face.

24. The computer readable medium of claim 23, wherein the projection of the second modulated light from the second light source off the optical axis of the modulated light camera does not result in a retinal reflection.

25. The computer readable medium of claim 24, wherein a portion of the light reflected from the face becomes non-modulated due to the phase difference between the first phase and the second phase.
